(19) United States
(12) Patent Application Publication
(10) Pub. No.: US 2007/0073364 A1
(43) Pub. Date: Mar. 29, 2007

(54) Combined OCT catheter device and method for combined optical coherence tomography (OCT) diagnosis and photodynamic therapy (PDT)

(75) Inventors: Oliver Meissner, Munich (DE); Martin Ostermeier, Buckenhof (DE); Martin Hoheisel, Erlangen (DE)

Correspondence Address: SCHIFF HARDIN LLP;Patent Department, 6600 Sears Tower, 233 South Wacker Drive, Chicago, IL, 60606, US

(73) Assignee: Siemens Aktiengesellschaft

(21) Appl. No.: 11/239,608

(22) Filed: Sep. 29, 2005

(51) Int. Cl. A61N 5/06; A61B 6/00

(52) U.S. Cl. 607/88; 600/476

(57) ABSTRACT

In a catheter device and a method for in vivo activation of a photosensitizing drug in a vessel, endovascular tissue, and/or intraluminal tissue, a catheter carrying both an optical coherence tomography (OCT) lens, from which OCT imaging light is emitted, and a photodynamic therapy (PDT) lens from which photosensitizing drug-activating light is emitted, is inserted into a vessel containing a lesion to be treated. A photosensitizing drug is caused to be placed in the vessel as well, such as in the form of a coating on a stent or a coating on an exterior of a balloon carried by the catheter. Light is emitted from the PDT lens to activate the photosensitizing drug while light is simultaneously emitted from the OCT lens to obtain an OCT image to monitor the drug activation.

### BACKGROUND OF THE INVENTION

1. Field of the Invention

The present invention is directed to a catheter device and a method that allow combined optical coherence tomography (OCT) diagnosis and photodynamic therapy (PDT).

2. Description of the Prior Art

Photodynamic therapy (PDT) is a therapeutic technique that makes use of light in combination with a photosensitizing drug. A photosensitizing drug is a drug that reacts chemically to light at an activation wavelength in the near-infrared, namely in a range between 664 to 1300 nm, in the presence of oxygen in order to destroy diseased or damaged cells. The photosensitive drug molecule is activated by light, causing conversion of oxygen molecules into toxic oxygen radicals (singlet oxygen). Singlet oxygen exists for less than a microsecond, but if prolonged light activation is performed at a sufficiently high rate, these oxygen radicals overcome the cell's natural defense, ultimately resulting in a highly localized tissue destruction.

PDT principles also may apply to underlying biochemical processes associated with pre-cancerous tissue changes or arteriosclerotic plaque.

A primary goal in the treatment of superficial endoluminal tumor invasion is to destroy the pathological tissue while sparing the surrounding healthy tissue. A primary goal in the treatment of atherosclerotic disease is the prevention of vascular tissue proliferation. A primary goal in the treatment of so-called “vulnerable plaques,” (lesions prone to rupture) is thickening of the fibrous cap that overlies the lipid core and/or prevention of neovascular leakage within the plaque.

Intravascular, intraluminal optical coherence tomography (OCT) is an imaging modality that provides histology-like cross-sectional images of vessels or hollow organs. The basic principles of OCT are well known, such as from PCT Application WO 97/0321282. With the high energy of near-infrared light, OCT is able to achieve diagnostic images of tissue with a spatial resolution of 10-20 μm. The near-infrared light has a similar energy spectrum to that necessary to activate photosensitizing drugs.

Conventionally, in the clinical context, PDT is routinely used in dermatology for treatment of skin lesions. Clinical studies are being conducted for ophthalmologic use of PDT (macula degeneration) as well as for gastrointestinal use (treatments of Barrett's esophagus). Intravascular treatment using PDT is under consideration, but thus far has not been the subject of clinical investigations. One reason for this is that in the treatment of skin lesions, for example, control of the PDT can be undertaken using ultrasound.

For optimization and precise usage of PDT intra-luminally or intravascularly, an imaging modality is needed that offers detailed information of the size, penetration depth, and structural changes of the lesion to be treated, such as pre-cancerous changes, tumor invasion, or arteriosclerotic plaques.

### SUMMARY OF THE INVENTION

It is an object of the present invention to provide a catheter device and a treatment method that allow precise, localized administration of PDT intraluminally or intravascularly.

The above object is achieved in accordance with the present invention in a method and a catheter device wherein an OCT catheter is provided with an additional light source to allow in situ (in vivo) PDT. Such a combination device and method for OCT diagnosis and PDT administration can be used in several ways.

In a first embodiment, a further lens/mirror system for PDT administration is added to an existing OCT catheter device. This further optical system must be placed at the catheter so that the therapy light is emitted in a direction opposite to the light emitted by the imaging lens/mirror system used to obtain the OCT image. The therapy energy should be matched to the photosensitizing drug that is being used, preferably 664 nm. This arrangement avoids the therapy light from interfering with the operation of the OCT system.

In another embodiment, an existing OCT catheter device can be optimized for using a single light source at an energy suitable both for diagnostic and therapeutic purposes.

OCT and PDT can be applied (operated) in alternation at a sufficiently high rate using the same optical system, in a further embodiment.

In another embodiment, a combined OCT/PDT system is constructed on a balloon catheter, the exterior of the balloon being coated with a photosensitive drug. The OCT probe can be used to investigate the vessel prior to therapy, and to activate the PDT during balloon insufflation under constant imaging.

The combined method and system according to the invention also can be used to activate a photosensitizing drug-coated stent after implantation and deployment of the stent.

The combined method and catheter device allows for immediate therapy monitoring, so that if necessary a repeated PDT can be administered.

Since OCT and PDT are performed with the same system, both procedures can be conducted in the same geometry (coordinate system). This allows easy coordination of the treatment planning with the therapy administration.

### DESCRIPTION OF THE DRAWINGS

FIG. 1 schematically illustrates a combined OCT/PDT catheter device in accordance with the invention, in an application with a stent coated with a photosensitizing drug.

FIG. 2 schematically illustrates a further embodiment of a combined OCT/PDT catheter device in accordance with the invention, used with a balloon catheter coated with a photosensitizing drug.

### DESCRIPTION OF THE PREFERRED EMBODIMENTS

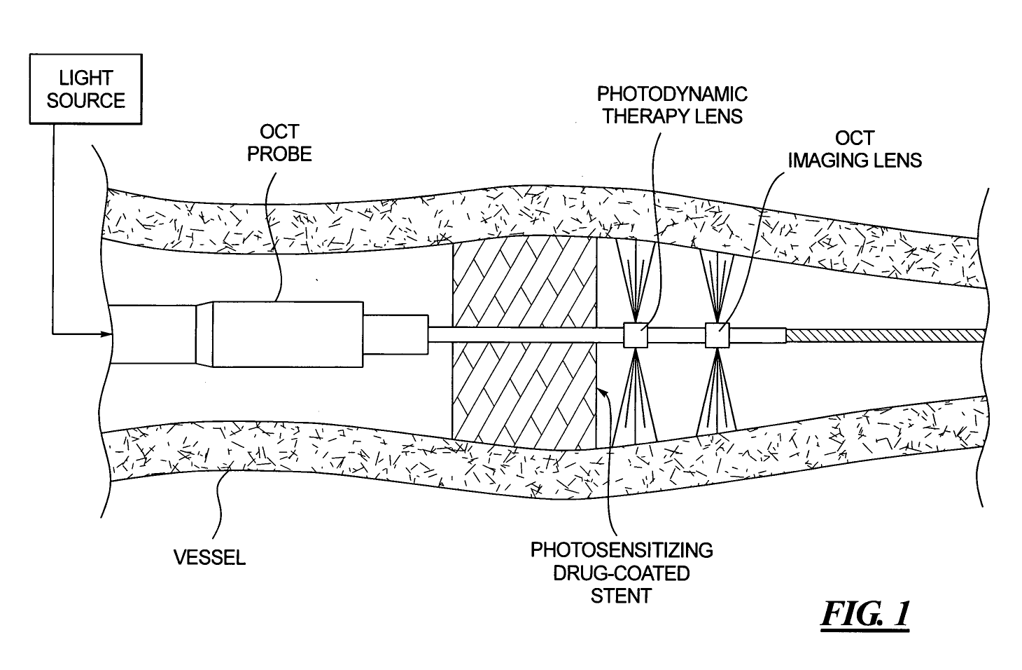

FIG. 1 schematically illustrates a combined OCT/PDT catheter device in accordance with the invention located in the interior of a vessel that, in this embodiment, has been stented with a photosensitizing drug-coated stent.

The catheter includes an OCT probe, operating in a known manner together with an OCT imaging lens to emit light to obtain an OCT image of the interior of the vessel.

In accordance with the invention, the catheter is also equipped with a photodynamic therapy lens from which PDT light is emitted to activate the photosensitizing drug on the stent. The drug can be used for treatment of vessel tissue, endovascular tissue, and/or intraluminal tissue. For clarity, the light emitted by the photodynamic therapy lens in FIG. 1 is shown next to the stent, however, in practice the catheter will be appropriately manipulated, such as in a pullback procedure, so that the light from the photodynamic therapy lens irradiates all of the surface of the stent that is coated with the photosensitizing drug, thereby to activate the photosensitizing drug at all locations. During this activation phase, real time imaging is obtained using the OCT imaging lens. OCT imaging can also be undertaken prior to the photosensitizing activation, using the OCT imaging lens, in a therapy planning stage, during which the PDT light is not activated.

As shown in FIG. 1, the OCT/PDT catheter is connected to a light source. The light source can contain a single source of light energy at a wavelength that is suitable both for OCT and PDT. Alternatively, the light source can contain two different light energy sources, one optimized for OCT and one optimized for PDT. These respective light energy sources can be operated (activated) in alternation at a high rate, so that neither the OCT imaging nor the PDT administration is impaired by the fact that the emitted light is non-continuous.

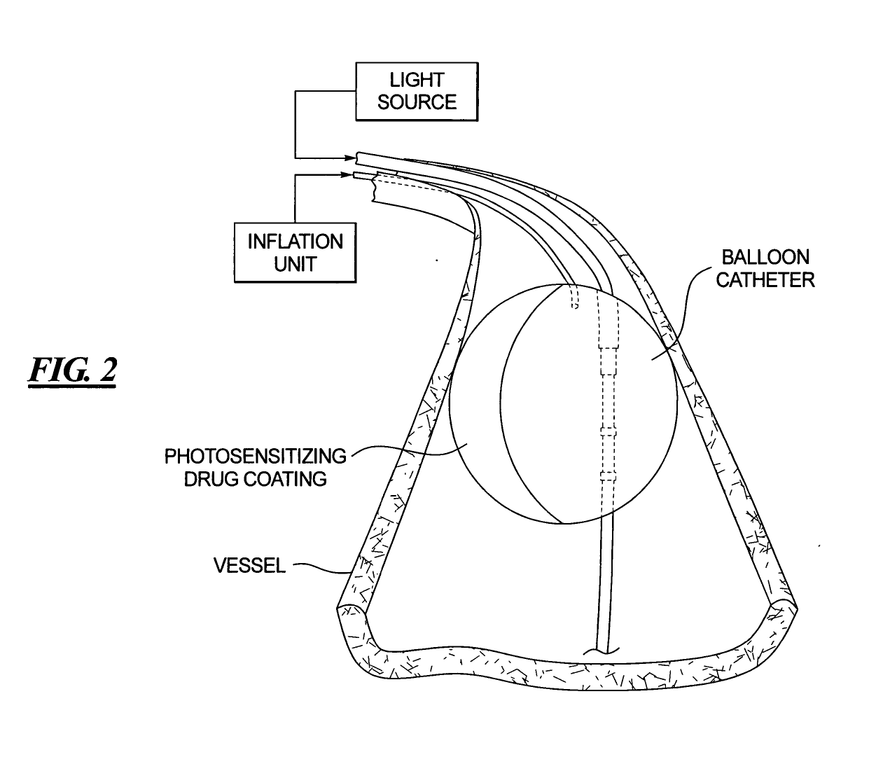

A further embodiment of a combined OCT/PDT catheter is shown in FIG. 2, in the form of a balloon catheter having a balloon with an exterior, or a portion of the exterior, coated with a photosensitizing drug. The portion of the catheter device inside the balloon is as shown in FIG. 1, and includes the OCT imaging lens and the photodynamic therapy lens described in connection with FIG. 1. After the balloon has been inflated by an inflation unit so that the exterior is in contact with the wall of the vessel, the PDT is administered by activating the PDT light that is emitted through the photodynamic therapy lens to activate the photosensitizing drug coating the exterior of the catheter. This PDT is monitored by simultaneous OCT, as described above in connection with FIG. 1. Moreover, before inflation of the balloon, therapy planning can be undertaken using OCT imaging as described in connection with FIG. 1.

In general, the inventive method and apparatus offer a combined diagnostic/therapeutic OCT/PDT device for intraluminal/intravascular PDT having the following advantages. Exact localization of the lesion that is to be treated can be obtained prior to PDT in the same coordinate system in which the PDT will be administered, making planning and monitoring of the PDT very simple. Using the OCT image, better delineation of the target lesion, and thus more precise therapy, can be obtained. Damage to surrounding tissue is thus minimized. The PDT itself is optimized, including selection of a light energy for the PDT that is best adapted to treat the lesion in question. In the event of a sub-optical therapeutic result, this can be noted by OCT monitoring, and an immediate repetition of the PDT, by reactivating the therapeutic light can be undertaken without removal and re-introduction of the catheter. Data, including imaging data, can be electronically acquired from the device during the entirety of the planning and therapy administration, allowing documentation of the procedure for study and archiving.

Although modifications and changes may be suggested by those skilled in the art, it is the intention of the inventors to embody within the patent warranted hereon all changes and modifications as reasonably and properly come within the scope of their contribution to the art.

We claim as our invention:

1. A catheter device comprising:
a catheter body;
an OCT probe and an OCT lens system carried on said catheter body, said OCT probe communicating with said OCT lens system through said catheter body;
a light source in optical communication through said catheter body with said OCT probe to cause said OCT lens system to emit OCT light;
a PDT lens system carried by said catheter body and being in optical communication through said catheter body with said light source to emit PDT light, said PDT lens system being carried by said catheter body at a location so that said PDT light does not interfere with OCT light; and
said catheter body and said OCT probe, said OCT lens system and said PDT lens system being configured for intraluminal/intra-arterial insertion.

2. A catheter device as claimed in claim 1 wherein said light source comprises a single source of light energy in optical communication with each of said OCT lens system and said PDT lens system.

3. A catheter device as claimed in claim 1 wherein said light source comprises a first source of light energy in optical communication with said OCT lens system and a second source of light energy in optical communication with said PDT lens system.

4. A catheter device as claimed in claim 3 wherein said light source operates said first source of light energy and said second source of light energy in alternation.

5. A catheter device as claimed in claim 3 wherein said second source of light energy emits light at a wavelength of 664 nm.

6. A stenting system comprising:
a stent configured for intraluminal/intra-arterial deployment, said stent having a photosensitizing drug coating thereon;
a catheter device comprising a catheter body an OCT probe and an OCT lens system carried on said catheter body, said OCT probe communicating with said OCT lens system through said catheter body a light source in optical communication through said catheter body with said OCT probe to cause said OCT lens system to emit OCT light a PDT lens system carried by said catheter body and being in optical communication through said catheter body with said light source to emit PDT light, said PDT lens system being carried by said catheter body at a location so that said PDT light does not interfere with OCT light, and said catheter body and said OCT probe, said OCT lens system and said PDT lens system being adapted for intraluminal/intra-arterial insertion; and
said catheter body being intraluminally/intra-arterially manipulatable to direct said PDT light from said PDT lens system onto said stent after deployment of said stent to activate said photosensitizing drug.

7. A stenting system as claimed in claim 6 wherein said light source comprises a single source of light energy in optical communication with each of said OCT lens system and said PDT lens system.

8. A stenting system as claimed in claim 6 wherein said light source comprises a first source of light energy in optical communication with said OCT lens system and a second source of light energy in optical communication with said PDT lens system.

9. A stenting system as claimed in claim 8 wherein said light source operates said first source of light energy and said second source of light energy in alternation.

10. A stenting system as claimed in claim 8 wherein said second source of light energy emits light at a wavelength of 664 nm.

11. A balloon catheter device comprising:
a catheter body;
an OCT probe and an OCT lens system carried on said catheter body, said OCT probe communicating with said OCT lens system through said catheter body;
a light source in optical communication through said catheter body with said OCT probe to cause said OCT lens system to emit OCT light;
a PDT lens system carried by said catheter body and being in optical communication through said catheter body with said light source to emit PDT light, said PDT lens system being carried by said catheter body at a location so that said PDT light does not interfere with OCT light;
a balloon carried on said catheter body with said OCT lens system and said PDT lens system contained in an interior of said balloon, said balloon having an exterior having at least a portion thereof coated with a photosensitizing drug;
an inflation unit in pressure communication with said interior of said balloon through said catheter body to inflate said balloon; and
said catheter body with said OCT probe, said OCT lens system, said PDT lens system and said balloon carried thereon being configured for intraluminal/intra-arterial insertion with said PDT light activating said photosensitizing drug on said exterior of said balloon after inflation of said balloon in a vessel.

12. A balloon catheter device as claimed in claim 11 wherein said light source comprises a single source of light energy in optical communication with each of said OCT lens system and said PDT lens system.

13. A balloon catheter device as claimed in claim 11 wherein said light source comprises a first source of light energy in optical communication with said OCT lens system and a second source of light energy in optical communication with said PDT lens system.

14. A balloon catheter device as claimed in claim 13 wherein said light source operates said first source of light energy and said second source of light energy in alternation.

15. A balloon catheter device as claimed in claim 13 wherein said second source of light energy emits light at a wavelength of 664 nm.

16. A method for in vivo activation of a photosensitizing drug, comprising the steps of:
providing a catheter carrying both an OCT lens system and a PDT lens system;
inserting said catheter into a vessel;
placing a photosensitizing drug in said vessel; and
activating said photosensitizing drug in said vessel by emitting activation light from said PDT lens system while simultaneously monitoring activation of said photosensitizing drug by obtaining an image with light emitted from said OCT lens system.

17. A method as claimed in claim 16 wherein the step of placing said photosensitizing drug in said vessel comprises deploying a stent in said vessel coated with said photosensitizing drug, and wherein the step of activating said photosensitizing drug comprises passing said catheter through an interior of said stent in said vessel while emitting said activation light from said PDT lens system.

18. A method as claimed in claim 16 wherein the step of placing said photosensitizing drug in said vessel comprises coating an exterior of a balloon with said photosensitizing drug and deploying and inflating balloon in said vessel with said catheter with said OCT lens system and said PDT lens system disposed in an interior of said balloon.

19. A method as claimed in claim 16 comprising emitting light from said OCT lens system and from said PDT lens system in alternation.
